(19) United States
(12) Patent Application Publication
(10) Pub. No.: US 2013/0006144 A1
(43) Pub. Date: Jan. 3, 2013

(54) BIOPSY NEEDLE WITH FLEXIBLE LENGTH

(76) Inventors: Michael Clancy, Monaleen Limerick (IE); Ciarán Toomey, Co. Cork (IE); Triona Campbell, Co. Clare (IE); Darach McGrath, Co. Tipperary (IE)

(21) Appl. No.: 13/525,984

(22) Filed: Jun. 18, 2012

Related U.S. Application Data
(60) Provisional application No. 61/502,139, filed on Jun. 28, 2011.

(51) Int. Cl. A61B 10/02

(52) U.S. Cl. 600/567

(57) ABSTRACT

An endoscopic tissue-sampling needle is provided including an elongate needle shaft having a proximal shaft portion and a distal shaft portion. The distal shaft portion extends into and is fixedly attached to an inner diameter of a proximal shaft portion lumen. The distal shaft portion lumen is configured for collection of patient tissue by including a distal penetrating tip and/or a side aperture with a cutting edge configured to excise tissue from a target site in a patient body. The proximal shaft portion includes a length of cable tube configured to provide enhanced flexibility in use via an endoscope.

### CROSS-REFERENCE TO RELATED APPLICATIONS

This application claims priority to U.S. Provisional Application Ser. No. 61/502,139, filed Jun. 28, 2011, which is incorporated herein by reference in its entirety.

### TECHNICAL FIELD

The invention relates generally to medical needles. More particularly, the invention pertains to medical needles configured for ultrasound-guided endoscopic biopsy.

### BACKGROUND

Endoscopists have developed great expertise in using elongate needles, including echogenic needles viewable under ultrasound, to obtain samples from patients in a minimally invasive manner. In particular, they use devices and techniques that allow carefully targeted collection of samples from deep in patient bodies without any external percutaneous incisions or punctures. Devices such as fine needle aspiration needles and fine needle biopsy needles may be directed through a working channel of an endoscope (e.g., duodenoscope, gastrointestinal end-viewing endoscope) to a target site in a patient body.

In order to obtain useful samples of tissue suitable for histological and/or cytological analysis, it is desirable to use a large-gauge needle. However, these needles are often considered stiff and unwieldy by some users who find them difficult to insert fully into, for example, a working channel of an endoscopic ultrasound (EUS) endoscope. In addition, as these needles typically include an outer sheath, it may be difficult to advance the penetrating/collecting distal end portion through and out of the sheath. These challenges may be particularly problematic when a user is attempting to access more difficult-to-reach anatomical locations (such as, for example, attempting to access the head of a patient's pancreas from the duodenum).

In addition, many needles (including, for example, those having stainless steel hypotube body) having a size useful for fine needle aspiration (FNA) may present other challenges that are related or different. Target sites may include hard-to-access locations such as, for example, the head of the pancreas, which—in minimally invasive procedures—may best be accessed through a side-viewing endoscope/duodenoscope via the duodenum or stomach. Patient anatomy can make it difficult to obtain samples from target sites and necessitate careful positioning and manipulation of an endoscope and needle(s). When used in an endoscope, the needle will often assume a shape set corresponding to the curvature of the torqued (i.e., actuated to effect curvature) portion of the endoscope's distal portion (see FIG. 1). When, as is sometimes the case, multiple needle passes are needed to get desired biopsy samples from one or more target sites, a user often will straighten out the needle after each pass to minimize the likelihood of its binding in the endoscope's working channel or otherwise interfering with easy operation of subsequent needle passes. This is inconvenient, time-consuming, and may lengthen the time needed to effect a biopsy procedure.

Another challenge associated with endoscopic needle procedures (whether FNA, FNB, or other) is described with reference to FIG. 1. Larger needles (e.g., 19 ga) are often desirable for obtaining larger FNA, FNB (fine needle biopsy), or other samples from a patient. Larger samples of cells, tissue, and/or other material may present diagnostic advantages (or even necessity) as compared to smaller samples obtainable from smaller (e.g., 25 ga) needles. However, as will be appreciated by those having skill in the art, larger gauge needles typically are stiffer, which limits the torqueability of an endoscope through which a needle is disposed.

FIG. 1 illustrates differences in ranges of motion afforded an endoscope by different needle gauges. An endoscope 12 was secured adjacent a flat comparison surface 10 and was loaded first with a 22 ga needle that was extended past the endoscope's elevator 13 and out of its working channel. The endoscope 12 was actuated to its maximum torqued/curved position, and its curvature (relative to a generally longitudinal axis of the scope marked as line 41) was marked with line 47. The maximal curved position of the 22 ga needle was marked as line 54 without the elevator 13 actuated, and as line 56 with the elevator fully actuated to position the needle at maximum curvature/angle relative to the endoscope.

The endoscope was then loaded with a 19 ga needle 14 that was extended past the endoscope's elevator 13 and out of its working channel. The endoscope 12 was actuated to its maximum torqued/curved position, and its curvature was marked with line 49. As also shown in FIG. 1, the maximal curved position of the 19 ga needle 14 was marked as line 64 without the elevator 13 actuated, and as line 66 with the elevator fully actuated to position the needle at maximum curvature/angle relative to the endoscope 12. As clearly shown by the relative positions of endoscope maximal curvature lines 47, 49, the use of a larger gauge needle clearly limits range of endoscope motion to a lesser curvature than allowed by a smaller-gauge needle. The difference in range of motion afforded the needle itself is markedly greater. Thus, a larger needle provides a limited range of motion, which limits a physician's ability to access certain anatomical structures/target sites with the needle. However, use of a smaller needle that allows access to harder-to-reach sites may require multiple passes to get a desired volume of sample(s). In addition to the potential need for manual needle-straightening noted above, this may increase procedure time and the costs attendant with physician/staff and facility time. In some circumstances these limitations may also have an impact on patient comfort and/or diagnostic efficiency.

It would therefore be advantageous to provide a needle that includes a distal end dimensioned to collect samples of a desirable size while also providing a shaft proximal of that distal end that obviates the present difficulties of advancement and navigation through an endoscope working channel and through patient anatomy to a variety of target site locations. The shaft provided should still provide desirable pushability and trackability so that the needle will be navigable in a manner consistent with the desires and aims of users to accurately obtain samples.

### BRIEF SUMMARY

In one aspect, an endoscopic tissue-sampling needle may be provided including an elongate needle shaft having a proximal shaft portion and a distal shaft portion. The proximal shaft portion includes a tubular body that includes a length of cable tube (which may be configured as a helical hollow strand material). The distal shaft portion may extend into and be fixedly attached to an inner diameter of a proximal shaft portion lumen. The distal shaft portion lumen preferably is configured for collection of patient tissue by including a distal penetrating tip and/or a side aperture with a cutting edge configured to excise tissue from a target site in a patient body. The proximal shaft portion may include a non-cable tube proximal sub-portion that is proximal of the cable tube portion.

### BRIEF DESCRIPTION OF THE DRAWINGS

FIG. 1 shows an endoscope and its limited range of motion during actuation with a needle disposed through its working channel;

FIG. 2 shows a needle device embodiment with a detail of a distal region thereof;

FIG. 3 shows a shaft length of an embodiment of a needle device;

FIG. 4 shows a distal portion of a needle device embodiment with adjacent cable tube portion; and

FIG. 5 shows a needle device embodiment with the distal needle portion including a side aperture.

### DETAILED DESCRIPTION

Embodiments are described with reference to the drawings in which like elements are generally referred to by like numerals. The relationship and functioning of the various elements of the embodiments may better be understood by reference to the following detailed description. However, embodiments are not limited to those illustrated in the drawings, and features of various embodiments—whether described in text and/or in drawing figures—may be incorporated into other embodiments within the scope of the present invention. It should be understood that the drawings are not necessarily to scale, and in certain instances details may have been omitted that are not necessary for an understanding of embodiments of the present invention, such as—for example—conventional fabrication and assembly.

As used in the specification, the terms “proximal” and “distal” should be understood as being in the terms of a physician or other person operating a medical device or on a patient. Hence, the term “distal means the direction or portion of the device that is farthest from the physician or other person and the term “proximal” means the portion of the device that is nearest to the physician or other person.

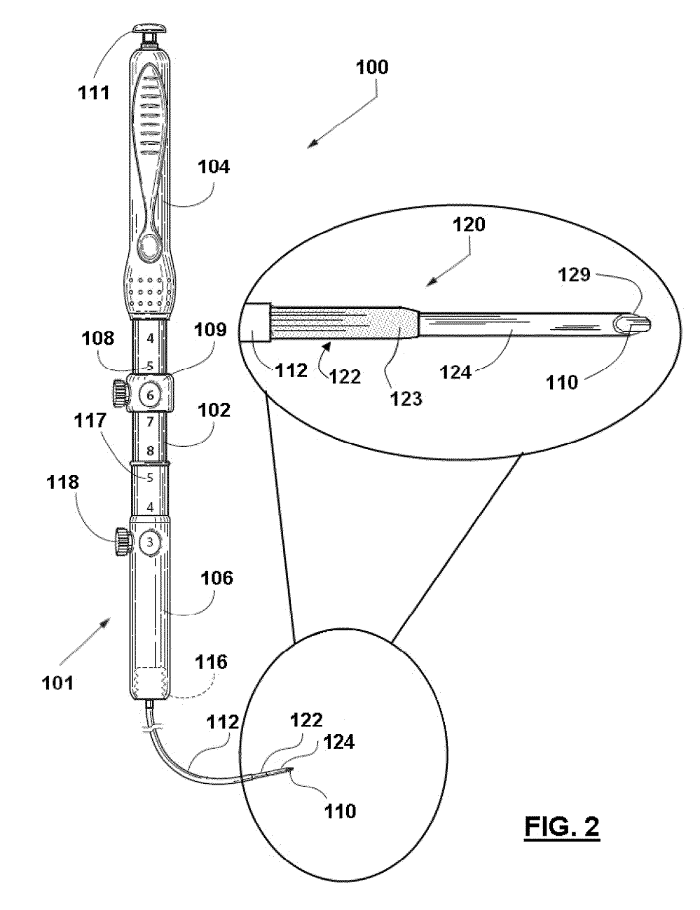

An endoscopic biopsy needle device 100, which may be scaled and configured for use in fine-needle aspiration (FNA) and/or fine-needle biopsy (FNB) procedures, or other tissue-collection procedures, is described with reference to FIG. 2. The needle device 100 is shown here with a handle 101. The handle 101 includes a sheath-attached handle member 102 with a needle-attached handle member 104 longitudinally slidably disposed on its proximal end. A scope-attachment handle member 106 is slidably attached to the distal end of the sheath-attached handle member 102. The sheath-attached handle member 102 is attached to the needle sheath 112 and the needle-attached handle member 104 is attached to the needle 120 (which may be configured in the manner of any of the needles disclosed herein or later developed in accordance with principles of the present disclosure). The sheath 112 may be constructed as a protective sheath configured to cover the needle 120 while it is being advanced through an endoscope working channel, which sheath will protect both the needle and the working channel from contact that could damage either or both.

The scope-attachment handle member 106 may be configured for incrementally fixable, longitudinally-adjustable (relative to the other handle components) attachment to the exterior of a working channel of an endoscope such as—for example—an end-viewing gastric endoscope, duodenoscope, or EUS endoscope (not shown) using, for example, a threaded cavity 116. The scope-attachment handle member 106 allows a user to determine the distance by which the sheath 112 will extend from a standard-length endoscope, and it may include numerical indicia 117 corresponding to that relative length and an adjustable engagement structure 118 allowing a user to select a length and engage the scope-attachment handle member 106 accordingly.

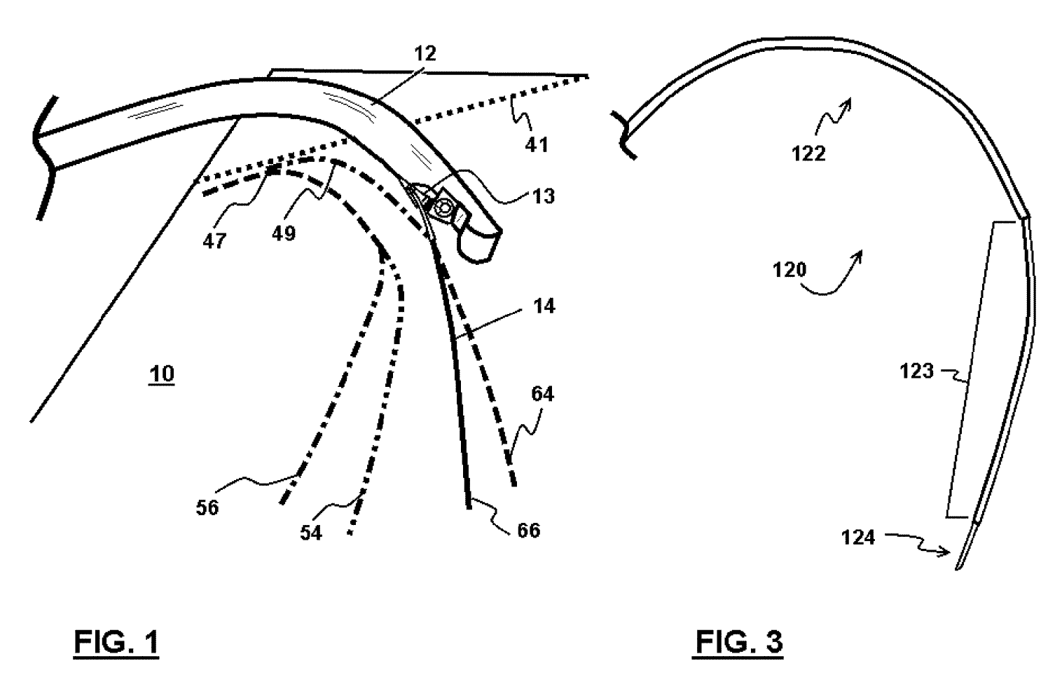

The sheath-attached handle member 102 includes numerical indicia 108 and an adjustable ring 109 that limits the movement of the needle-attached handle member 104 and provides a way to select the distance to which the needle 120 may be extended beyond the sheath 112. By way of illustration, the configuration shown in FIG. 1 would allow the sheath to extend 3 units (e.g., inches, cm) beyond the distal end opening of an endoscope working channel, and the needle 120 would be allowed to extend up to 6 units beyond the distal end of the sheath 112, although its current position would be only about 4 units beyond the distal end of the sheath 112 (based upon the position shown of the needle-attached handle member 104). A stylet 110 extends through a lumen of the needle 120 and has a stylet cap 111 fixed on its proximal end. It should be appreciated that other embodiments of the handle described herein, as well as other handle designs appropriate for use in operating a biopsy needle may be practiced within the scope of the present invention.

FIG. 1 includes a detail call-out showing the distal portion of the needle device 100. The needle 120 extends distally beyond the distal end 129 of the sheath 112. As is described below in greater detail, the needle 120 includes two sections—a proximal portion 122 (including a coated cable tube portion 123), and a distal portion 124. A stylet 110 is shown extending from the distal end of the needle distal portion 124. The stylet 110 is shown as a round-tipped stylet, but it should be appreciated that other stylet designs, including a stylet having a distal end tip beveled or otherwise shaped to match or otherwise complement a distal tip shape and/or geometry of the needle 120.

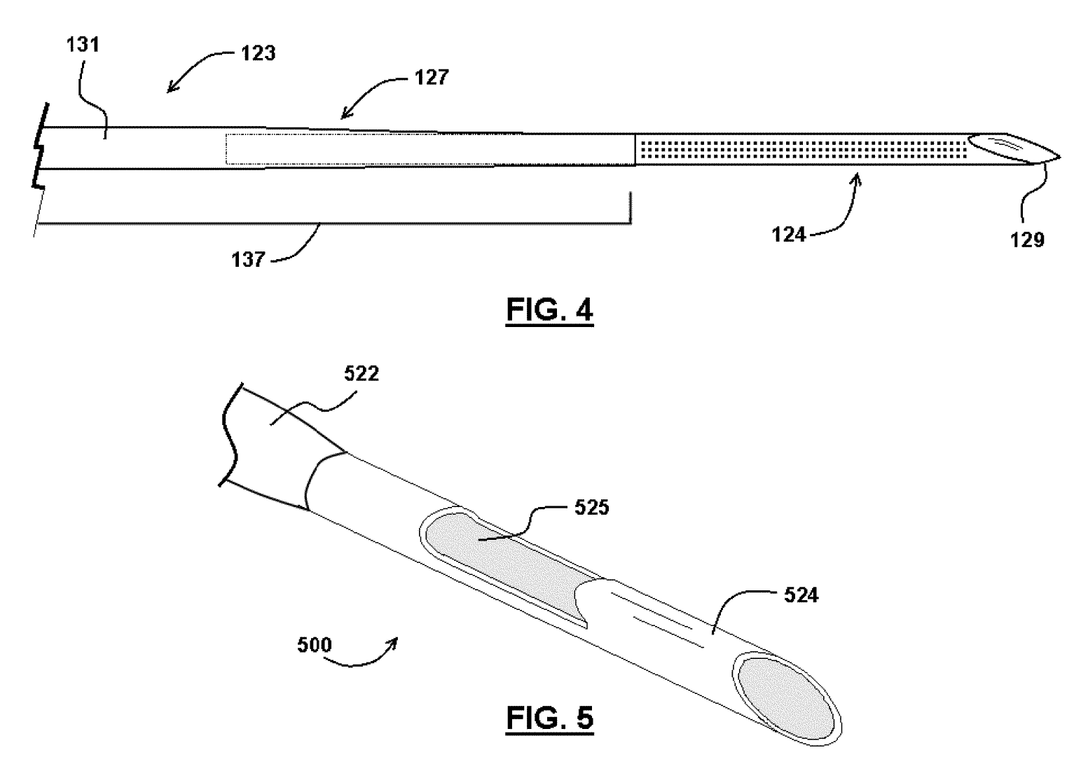

FIG. 3 shows perspective view of a length of the needle 120, and FIG. 4 shows a more detailed view of a distal region of the needle device embodiment. Certain embodiments configured for use with a stylet may be configured with greater flexibility, as the stylet can be used to provide desirable stiffness, pushability, and trackability when navigating the needle to a target site in a patient body. The distal needle portion 124 includes a distal tip 129, which is shown as a beveled needle tip. It should be appreciated that the distal tip 129 preferably is configured for collecting a tissue sample (e.g., suitable for cytological and/or histological analysis), and may be configured in a variety of ways including with different penetrating needle tip styles known or developed in the art including, for example, Chiba, Franseen, Menghini, Turner, and/or other needle tip types, or it may be constructed as an atraumatic tip (e.g., as in the Cook® ECHO-19-A device). The needle shown may include a needle lumen through which a stylet may be disposed.

The distal needle portion 124 will generally be much shorter than the proximal needle portion 122. The distal needle portion 124 will preferably be about 20 mm to about 40 mm in length, although some embodiments may be about 100 mm in length or more. In many embodiments configured for use with endoscopic pancreatic biopsy, it will often be preferable that the distal needle length not be much greater than about 40 mm, as it may then occupy a portion of the endoscope that needs to flex more during a procedure than may be permitted by the needle cannula. The total length of the needle 120 preferably will be configured to access a target site in a patient site via an endoscope (e.g., about 100 cm to about 180 cm or greater, exclusive of a handle). Preferred needle designs often will include echogenicity-enhancing features such as, for example, surface dimples, laser etching, grit-blasting, or other structures configured to provide desirable ability to visualize the needle under ultrasound, including endoscopic ultrasound. A pattern of dimples is shown on the surface of the distal needle portion 124 in FIG. 4.

The proximal needle portion 122 may mostly be configured as stainless steel hypotube or another traditional cannula material, or it may include an intermediate region 123 that is formed of cable tubing 131. The cable tubing used may include one or more of coiled, multifilar, woven, stranded, braided, and/or crosswound configuration(s) construction(s). Cable tubing of these and other configurations may be sterilized and used in medical devices with great effect

One particularly preferred cable tubing is Helical Hollow Strand (HHS™) from Fort Wayne Metals (Fort Wayne, Ind.). HHS™ is a stranded wire with an open center working channel that provides highly flexible tubing, which generally will not assume a shape set in the same manner as hypotube or other cannula materials. Additionally, as a feature of its great flexibility, HHS™ is highly crimp-resistant and kink-resistant, and it provides a high degree of pushability and trackability.

In some embodiments, a major length or the entire proximal needle portion 122 may be configured as cable tubing. Generally, the metallic cannula length distal of (and—in some embodiments—proximal of) the cable tube length will include lower flexibility, lower resilience, or both relative to the cable tube length.

FIG. 4 provides a detailed view of the distal needle portion 124 where it joins the proximal portion 122. The proximal end of the distal needle cannula 124 is inserted into the lumen of the cable tubing 129. The overlap region 127 may be, for example, about 1 to about 2 cm and may be secured there by adhesive, soldering, and/or welding. As shown in FIG. 4, a larger outer diameter cable tube may be ground or otherwise shaped to form a reduced outer diameter transition to a smaller diameter needle and prevent a minimally traumatic/substantially atraumatic outer profile. Heat shrink or another form of polymer or other coating may be applied to the inner and/or outer diameter of the cable tube section to provide a seal. For example, the heat shrink or other coating may extend along a length 137, as shown in FIG. 4, to provide the cable tube length with a fluid-tight seal. This may be important if the needle is to be used for injection, and/or if it needs to maintain a vacuum (e.g., for FNA, FNB). The coating may also be configured as (or include) a low-friction material.

The cable tube 129 preferably extends proximally from the distal needle 124 for at least about 8 cm, or another length sufficient to traverse the actively flexing distal portion of an endoscope with which the needle device will be used, in any embodiment where it is configured as an intermediate portion 123. As noted above, the cable tube 129 may extend for the entire length of proximal needle portion 124. In embodiments where the cable tube length 129 is configured as an intermediate portion 123, its proximal end may be secured to a proximal length of metal cannula by adhesive, welding, and or soldering. In many circumstances, it may be most economical to provide the cable tube only for an intermediate length, with the needle cannula 124 distal thereof and a proximal metal, polymer, or other material tube length proximal thereof. The overall length and flexibility preferably are such that the device is deployable through a working channel of an endoscope such as, for example, a side-viewing gastrointestinal endoscope.

Some users of endoscopic needles with traditional metallic shaft bodies have observed that they may be difficult to advance fully into/through an EUS endoscope. A cable tube intermediate region 123 may obviate this by providing a shaft with good pushability that will include greater flexibility than traditional metallic cannula shafts of similar gauge, thereby decreasing potential binding in the endoscope working channel. For example, the cable tube may include stiffness, pushability, and/or trackability comparable to a 22 ga or even a 25 ga stainless steel endoscopy needle (e.g., as shown in FIG. 1), while providing—for example—a 19 ga distal needle end for interacting with tissue. This combined cable tubing plus distal needle structure will provide the ability to obtain desirable biopsy samples using a 19 ga or possibly even larger gauge needle, while simultaneously providing the flexibility and control previously available only with much smaller gauge needles. In addition, unlike traditional needles using metallic cannulas, designs in keeping with the present embodiments will not assume a shape set or other bending, curving, or crimping from being used through an endoscope with its distal end fully actuated. Furthermore, embodiments as described herein will be unlikely to limit the range of motion of an endoscope in the manner associated with larger gauge traditional metal cannulas, regardless of the distal needle gauge being used.

The proximal region 122 may be constructed of metallic tubing in the manner of existing endoscopic needles, the same or a different cable tube material as the intermediate region 123, a polymeric tubing, a coated or uncoated metallic tubing, or other suitable material known in the art. The outer diameter of the intermediate region 123 may be the same, greater than, or less than the outer diameters of the proximal region 122 and the distal region 124, which may be the same or different than each other. An embodiment with a cable tube intermediate portion 123 may provide a proximal portion with greater rigidity (e.g., where stainless steel cannula is used), an intermediate portion with greater flexibility (than either the proximal or end portion) while retaining desirable pushability, and a distal end needle portion configured to penetrate or otherwise interact with a target region accessible via an endoscope. The enhanced flexibility of the intermediate portion over current devices may provide advantages in accessing anatomical locations that are not readily accessible to less flexible metal-body cannulas used in many current endoscopic echogenic needle devices, while providing the ability to use a larger gauge needle than could otherwise be used to access those less-accessible sites.

The length of a cable tube intermediate region 123 may be relatively short or long in comparison to the overall needle length. For example, in a gastrointestinal endoscopy needle of about 180 to about 320 cm in length, the cable tube section may be only about 40 to about 320 mm in length, although the length may be greater or less. For example, in one embodiment of a needle, the intermediate polymer section of a 240 cm needle device is only about 80 mm in length, with a distal metal needle end that is 26 mm in length. However, the distal metal needle length may be greater or less than about 10 mm. This generally distal location of the enhanced-flexibility cable tube device length may provide desirable flexibility along the portion most likely to be directed through restricted, tortuous, or otherwise difficult-to-navigate paths (e.g., in and/or exiting an endoscope working lumen, in a patient body lumen, extending through body tissue) in addition to preferably being optimally located for occupying the distal coiling/curving actuatable length of an endoscope. In this and other embodiments, larger-outer-diameter more proximal shaft lengths may be located/configured where they will not penetrate tissue, but will remain in an endoscope working lumen or open body lumen.

FIG. 5 shows another embodiment of a needle device 500. The device includes a proximal cable tube portion 522 that is coated and tapered to where it is attached to a distal needle portion 524. The needle portion 524 includes a notch or other aperture 525 in its sidewall, which may provide for and/or enhance certain sample collection methods.

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment of the invention.

We claim:

1. An endoscopic tissue-sampling needle, comprising:
an elongate tubular needle shaft having a proximal shaft portion and a distal needle portion;
wherein the distal needle portion comprises a shorter length than the proximal shaft portion;
wherein a length of the proximal shaft portion comprises a length of cable tube disposed immediately proximally adjacent of and securely fixed to the distal needle portion, said cable tube length comprising a greater flexibility than the distal needle portion;
wherein the distal needle portion
comprises a metallic material;
is configured for collection of patient tissue; and
wherein the elongate tubular needle shaft is configured and dimensioned for passage through a working channel of an endoscope to a target site within a patient body, with the cable tube length configured to occupy a curvably actuatable portion of an endoscope.

2. The needle of claim 1, further comprising a metallic cannula length disposed immediately proximally adjacent of and securely fixed to the cable tube length, where the metallic cannula length includes lower flexibility, lower resilience, or both relative to the cable tube length.

3. The needle of claim 1, wherein the cable tube length comprises a substantially uniform outer diameter that is larger than the outer diameter of the distal needle portion and that tapers proximally-to-distally to an outer diameter that is about the same as the distal needle portion outer diameter, which distal needle outer diameter is substantially uniform along an entire length of the distal needle portion.

4. The needle of claim 1, wherein the cable tube length comprises an outer diameter that is substantially the same as the outer diameter of the distal needle portion, such that the elongate tubular needle shaft comprises a substantially uniform outer diameter along an entire length thereof.

5. The needle of claim 1, wherein the distal shaft portion comprises a tissue-penetrating distal end tip.

6. The needle of claim 1, wherein the distal shaft portion comprises a notched aperture in at least one side.

7. The needle of claim 6, wherein the notched aperture comprises at least one cutting edge configured to enhance collection of sample material from a patient body.

8. The needle of claim 1, wherein the distal shaft portion is about 20 mm to about 100 mm in length.

9. The needle of claim 1, wherein the proximal shaft portion includes a proximal shaft lumen, the distal shaft portion includes a distal shaft lumen, and the proximal and distal shaft lumens together provide a continuous shaft lumen.

10. The needle of claim 1, wherein the distal needle portion comprises at least one surface feature configured to enhance echogenicity.

11. The needle of claim 10, wherein the stylet further comprises a third outer diameter length that is disposed distal of the second outer diameter distal length, where the third outer diameter is greater than the second outer diameter.

12. The needle of claim 1, wherein the distal needle portion measures no less than about 19 gauge.

13. The needle of claim 1, wherein the distal needle portion measures about 19 gauge.

14. The needle of claim 1, wherein the cable tube portion comprises a helical hollow strand configuration.

15. The needle of claim 1, further comprising a metallic cannula length disposed immediately proximally adjacent of and securely fixed to the cable tube length, where the cable tube length is about 8 cm.

16. The needle of claim 1, wherein the cable tube length comprises one or more of a coiled, multifilar, woven, stranded, braided, and crosswound configuration.

17. An endoscopic tissue-sampling needle, comprising:
an elongate tubular needle shaft having a proximal shaft portion comprising a first metal cannula and a distal needle portion comprising a second metal cannula;
wherein the distal needle portion comprises a shorter length than the proximal shaft portion;
a length of cable tube disposed intermediate the first and second metal cannulas, wherein a distal end of the cable tube is immediately proximally adjacent of and securely fixed to the distal needle portion, said cable tube length comprising a greater flexibility than the distal needle portion;
wherein the distal needle portion is configured for collection of patient tissue; and
wherein the elongate tubular needle shaft is configured and dimensioned for passage through a working channel of an endoscope to a target site within a patient body, with the cable tube length configured to occupy a curvably actuatable portion of an endoscope.

18. The needle of claim 17, wherein a combined length of the proximal shaft portion and the distal shaft portion is configured to access tissue via passage through at least a patient esophagus and stomach.

19. The needle of claim 17, wherein the distal needle portion is configured as a 19 gauge needle.

20. A method of making a needle according to claim 17, said method comprising steps of:
providing a length of cable tube, a length of the first metal cannula, and the second metal cannula configured as a distal needle end of about 20 to about 80 mm in length;
affixing a distal end of the cable tube to the second metal cannula by a select one or more of adhesive, soldering, and welding; and
affixing a proximal end of the cable tube to the first metal cannula by a select one or more of adhesive, soldering, and welding.
